(19) United States
(12) Patent Application Publication
(10) Pub. No.: US 2026/0357323 A1
(43) Pub. Date: Nov. 12, 2026

(54) DETECTION OF EPIGENETIC MODIFICATIONS

(71) Applicant: Roche Molecular Systems, Inc., Pleasanton, CA (US)

(72) Inventors: Frank Bergmann, Iffeldorf (DE); Dieter Heindl, Muenchen (DE)

(21) Appl. No.: 19/103,070

(22) PCT Filed: Aug. 16, 2023
(86) PCT No.: PCT/EP2023/072497

§ 371 Date: Feb. 11, 2025

(30) Foreign Application Priority Data
Aug. 18, 2022 (EP) 22191052.4

(51) Int. Cl. C12Q 1/6806

(52) U.S. Cl. CPC C12Q 1/6806; C12Q 2600/154

(57) ABSTRACT

The present invention provides a method comprising the steps of a) providing a nucleic acid comprising 5fC, 5hmC, or 5caC, b) providing one reactant comprising two reactive groups wherein the first reactive group is capable of reacting with the formyl hydroxymethyl or carboxyl group, and the second reactive group is a nucleophilic group, c) reacting said first reactive group with the formyl, hydroxymethyl or carboxyl group thereby resulting in a modified 5fC, 5hmC, or 5caC, d) reacting said second reactive group with the C6 position of said modified 5fC, 5hmC, or 5caC, thereby obtaining a bicyclic or tricyclic molecule comprising a 5,6-di-hydro Cytosine entity, and e) deaminating said 5,6-di-hydro Cytosine entity to a 5,6 di-hydro-Uracil entity.

### FIELD OF THE INVENTION

The invention relates to the field mapping the epigenetic modifications of DNA and RNA, which has become increasingly important, because these modifications play a role in several biological processes and diseases including development, aging and last but not least, cancer.

### TECHNICAL BACKGROUND

Most relevant to identify epigenetic modifications is detection by sequencing based methods. Differentiation between cytosine (C) and 5-methylcytosine (5mC) is described by direct sequencing (without preamplification) of DNA through a nanopore (e.g. by Pacific Bioscience Single Molecule, Real-Time (SMRT) Sequencing technology) and thereby reading out the different kinetics of nucleotide incorporation opposite C vs. mC by polymerase.

Other techniques are using methods to convert C or mC into a T(U) equivalent and subsequent amplification to allow identification when comparing untreated and converted sample DNA (see review: L. Zhao et al., Protein Cell 2020, 11, 792-808).

Most important methods are bisulfite sequencing (conversion of C to U by bisulfite treatment), NEB's EM-seq method (oxidation of mC by TET2 enzyme and glucosylation by β-glucosyltransferase (to block enzymatic deaminase reaction) and convert C to U by APOBEC deaminase), TAPS method (TET-assisted pyridine borane sequencing) applying TET enzyme oxidation of mC and subsequent reduction of oxidized mC species by pyridine borane to obtain dihydrocytosine nucleoside which is easily deaminated to give dihydrouracil (T equivalent) and the CLEVER method which is based on oxidation of mC by TET enzyme and subsequent reaction of 5-formyl-C with malononitrile to give an adduct which acts predominantly as a T equivalent in subsequent PCR amplification (C. Zhu et al., Cell Stem Cell 2017, 20, 720-731). Instead of enzymatic oxidation of mC by TET enzymes oxidation can also be performed by chemical means using e.g. potassium perruthenate (KRuO₄).

There are also methods to differentiate the modifications 5-hydroxymethyl-dC (5hmC), 5-formyl (5 fC) or 5-carboxyl-C (5caC) by partly modifying the methods described above.

Recently, WO 2022/096751 also disclosed a method for generating a di-hydrothymine (DHT) or a dihydrouracil (DHU) residue from a nucleoside or a polynucleotide containing 5-methylcytosine (5mC) or 5-carboxyl-cytosine (5caC) by using a radical initiator which may be used together with a nucleophilic compound.

However, all the methods disclosed have some shortcomings. Bisulfite sequencing encompasses harsh conditions which degrades the majority of input DNA, only indirect detection of methylated cytosines is possible. Likewise, Enzymatic Methyl Sequencing (EM-Seq) via enzymatic deamination which is inefficient because the majority of cytosines which are non-methylated have to be converted. TAPS requires a toxic reagent needed for reduction such as pyridine borane, and the dihydrouridine being labile under slightly acidic conditions cannot easily be converted back to the more stable Uridine derivative. For the CLEVER method, the conversion yield of the condensation reaction of 5fC and malononitrile is far below 100%, because the enzymatic incorporation of nucleotides by polymerase when the condensation product of 5fC with malononitrile acts as substrate is not highly accurate, and incorporation of dA is only favoured, but not exclusive (see also: F. Galardi et al., Biomolecules 2020, 10, 1677). The method of WO 2022/096751 is not entirely specific for caC but also converts unmodified C to some extent. Another disadvantage is that di-hydro Uridine derivative cannot easily be converted back to the more stable Uridine derivative.

### SUMMARY OF THE INVENTION

The present invention is based on the basic idea that the introduction of a nucleophile at 5fC, 5hmC or caC and the subsequent intramolecular addition at C-6 of a 5-substituted cytosine is enabling a deamination at C-4 of a cytosine derivative.

Thus, in a first aspect, the present invention is directed to a method comprising the steps of
a) providing a nucleic acid comprising 5fC, 5hmC, or 5caC
b) providing one reactant comprising two reactive groups wherein the first reactive group is capable of reacting with the formyl, hydroxymethyl, or carboxyl group, and the second reactive group is a nucleophilic group,
c) reacting said first reactive group with the formyl, hydroxymethyl or carboxyl group thereby resulting in a modified 5fC, 5hmC, or 5caC
d) reacting said second reactive group with the C6 position of said modified 5fC, 5hmC, or 5caC, thereby obtaining a bicyclic or tricyclic molecule comprising a 5,6 di-hydro Cytosine entity, and
e) deaminating said 5,6 di-hydro Cytosine entity to a 5,6 di-hydro-Uracil entity.

The newly formed cyclic structures in step d) are 5-7 membered rings

In one embodiment, the method further comprises step f), i.e. reversing the ring formation of step d) thereby obtaining a 5-substituted Uracil.

Since the naturally occurring methylation of DNA is 5mC, the method may also comprise a step of converting 5mC to 5fC, 5hmC or 5caC prior to step a). Thus, the present invention is facilitating the methylation status analysis of naturally occurring DNA.

In a specific embodiment, said nucleic acid comprises at least one 5fC residue. In this case, the first reactive group may be a C-H acidic group, an amine or a phosphorus ylide. In another specific embodiment, said nucleic acid comprises at least one 5hmC residue. Then, the first reactive group may constitute a Glycosyl Transferase substrate. In still another embodiment, said nucleic acid comprises a 5caC residue. Then, the first reactive group may be an amine.

In all cases, the said second reactive group may be a nucleophilic group selected from a group consisting of Thiol or Sulfinate.

All methods as disclosed above may further comprise a subsequent step of amplifying said nucleic acid, which may be preferably executed by means of PCR amplification. As well, all methods disclosed above may further comprise the step of a subsequent step of sequencing said nucleic acid either with or without a prior amplification step, which is preferably a PCR amplification.

### BRIEF DESCRIPTION OF FIGURES

FIG. 1 provides a scheme for detection of methylated sequences according to the present invention.

FIG. 2 shows a reaction scheme for modification of 5fC by means of a Knoevenagel condensation using a reactant with a C-H acidic group

FIG. 3 shows a reaction scheme for modification of 5fC by means of an aldol condensation reaction with a CH-acidic reagent comprising a sulfinate moiety

FIG. 4 shows a reaction scheme for modification of 5fC by means of a Wittig reaction.

FIG. 5 shows a specific example for a Wittig reaction according to FIG. 4 with a sulfinate modified Wittig reagent.

FIG. 6 shows a reaction scheme for modification of 5fC by means of reductive amination

FIG. 7 shows a specific example for a reductive amination as in FIG. 6, where 5fC is reacted with 2-aminomethyl sulfinate

FIG. 8 shows a reaction scheme for the modification of hmC using UDP-glucose substituted at the 2-position of the glucose moiety with a nucleophile as a reagent and executing a subsequent beta-glucosyl-transferase reaction.

FIG. 9 shows a similar reaction scheme as example 8, wherein 2-thio-glucose-UDP is used.

FIG. 10 shows an example for modification of the carboxyl group of caC.

### DETAILED DESCRIPTION OF THE INVENTION

### ABBREVIATIONS

C—cytosine or cytidine
T—thymine or thymidine
U—uracil or uridine
DHU—dihydrouracil or dihydrouridine
5mC—5-methylcytosine or 5-methylcytidine
5hmC—5-hydroxymethyl cytosine or 5-hydroxymethyl cytidine
5fC—5-formylcytosine or 5-formylcytidine
5caC—5-carboxylcytosine or 5-carboxylcytidine
dC—2′-deoxycytidine
dU—2′-deoxyuridine
TET—ten-eleven translocation dioxygenase
TAPS—TET-assisted pyridine-borane sequencing
CAPS—chemically-assisted pyridine-borane sequencing

### DEFINITIONS

A nucleophilic group is a functional group that is attractive to electron-poor or positively charged centers and donates electrons, in particular donating an electron pair to an electrophile or an electrophilic center to form a covalent bond. Nucleophilic groups are for example alcohols, alcoholates, thiols, thiolates, amines, sulfinates or carbanions. The term deamination refers herein to a substitution reaction of an exocyclic amino group by a hydroxyl group, in particular substitution of the amino group at the C-4 position of a cytosine nucleobase to convert into a uracil nucleobase. Deamination at C-4 position is enhanced in 5,6-dihydrocytosines to give 5,6-dihydrouracil derivatives, as e.g. being applied after bisulfite addition to the C5-C6 double bond of cytosine.

The term C-H acidic group is used herein for a carbon atom within a compound which carries at least one electron-withdrawing group and thus being more easily deprotonated by a base. Electron-withdrawing groups are for example carbonyl containing groups, a nitro group, a cyano group, a sulfoxide or sulfone group.

The term Wittig reagent or phosphorus ylide, as used herein, refers to neutral dipolar molecules containing a formally negatively charged carbon atom (a carbanion) directly attached to a phosphorus atom with a formal positive charge. In phosphorus ylides (phosphonium ylides) the two adjacent carbon and phosphorus atoms are connected by both a covalent and an ionic bond. Phosphorus ylides are thus 1,2-dipolar compounds and a subclass of zwitterions. In a Wittig reaction triphenylphosphonium ylides are reacted with aldehydes or ketones to give an alkene bond. In a Horner-Wadsworth-Emmons reaction stabilized phosphonate carbanions are used as reagent.

A Glycosyl Transferase Substrate, as used herein, refers to a reagent comprising an activation group, e.g. a UDP moiety, and a sugar residue, for example a glucose or substituted glucose. Glycosyltransferases catalyze the transfer of the sugar moiety from an activated donor sugar (the glycosyl transferase substrate) onto a saccharide or non-saccharide acceptor. The non-saccharide acceptor can be for example 5-hydroxymethyl-dC. T4 Phage β-glucosyltransferase, for example from New England Biolabs, specifically transfers a glucose moiety of uridine diphosphoglucose to the 5-hydroxymethylcytosine (5-hmC) residues in double-stranded DNA, making beta-glucosyl-5-hydroxymethylcytosine.

### Methods for DNA Methylation Status Analysis by Means of Modification, Followed by Sequencing

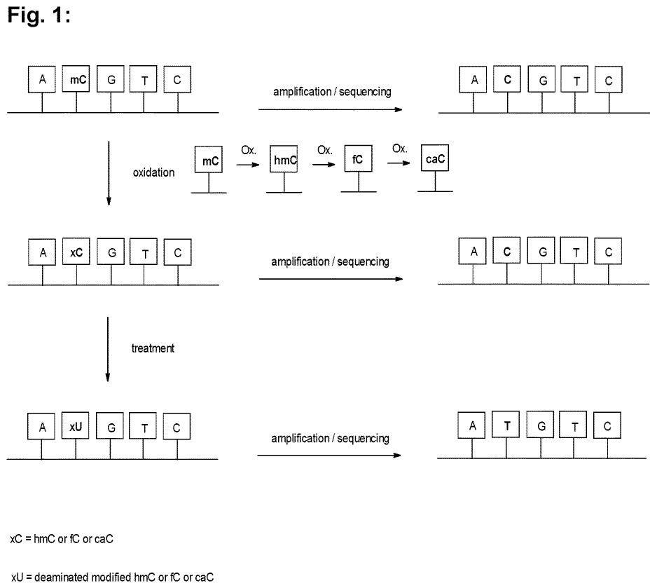

A general scheme, how methylated C-residues mC are detected is schematically shown in FIG. 1. Without any particular treatment, the epigenetic dC modifications 5-methyl-dC (mC), 5-hydroxymethyl-dC (hmC), 5-formyl-dC (fC) and 5-carboxyl-dC (caC) are read as unmodified dC when amplified and/or sequenced. However, oxidation of mC by enzymatic and/or chemical means results in oxidized mC modifications hmC, fC and caC (general: xC).

Oxidation or conversion of 5mC to 5 fC can be achieved by using TET enzymes (ten-eleven translocation (TET) methylcytosine dioxygenases). The same class of enzymes are suitable for converting 5mC to 5 hmC. 5 hmC can be further oxidized to 5fC enzymatically by a laccase enzyme or chemically using either KRuO4 or Cu(II)/TEMPO. Further details are disclosed in Pfeifer et al. Epigenetics & Chromatin 2013, 6:10, p. 1-9.

Subsequently, 5fC, 5hmC or caC are reacted with a reagent of the invention comprising two reactive groups, the first reactive group forming a covalent bond with hmC, fC or caC and thus introducing the second reactive group which comprises a nucleophile. By intramolecular addition of the nucleophile to the C5-C6 double bond of hmC, fC or caC deamination at C4 position is initiated resulting in a modified dU derivative which is read in a subsequent amplification or sequencing step as a T, thus enabling detection of epigenetic modification.

Such a sequencing according to the present invention allows the generation of data in the methylation status of the DNA of interest. Thus, the present also provides a method for analyzing the methylation status of a naturally occurring DNA comprising the steps of
a) converting 5mC to 5fC, 5hmC or 5caC
b) providing one reactant comprising two reactive groups wherein the first reactive group is capable of reacting with the formyl, hydroxymethyl or carboxyl group, and the second reactive group is a nucleophilic group,
c) reacting said first reactive group with the formyl, hydroxymethyl or carboxyl group thereby resulting in a modified 5fC, 5hmC, or 5caC,
d) reacting said second reactive group with the C6 position of said modified 5fC, 5hmC, or 5caC, thereby obtaining a bicyclic or tricyclic molecule comprising a 5,6 di-hydro Cytosine entity, and
e) deaminating said 5,6 di-hydro Cytosine entity to a 5,6 di-hydro-Uracil entity.

In some embodiments, the sample is derived from a subject or a patient. In some embodiments the sample may comprise a fragment of a solid tissue or a solid tumor derived from the subject or the patient, e.g., by biopsy. The sample may also comprise body fluids that may contain nucleic acids (e.g., urine, sputum, serum, blood or blood fractions, i.e., plasma, lymph, saliva, sputum, sweat, tear, cerebrospinal fluid, amniotic fluid, synovial fluid, pericardial fluid, peritoneal fluid, pleural fluid, cystic fluid, bile, gastric fluid, intestinal fluid, or fecal samples). In other embodiments, the sample is a cultured sample, e.g., a tissue culture containing cells and fluids from which nucleic acids may be isolated. In some embodiments, the nucleic acids of interest in the sample come from infectious agents such as viruses, bacteria, protozoa or fungi. The present invention involves manipulating isolated nucleic acids isolated or extracted from a sample. Methods of nucleic acid extraction are well known in the art (see, Sambrook et al., & quot; Molecular Cloning: A Laboratory Manual, & quot; 1989, 2nd Ed., Cold Spring Harbor Laboratory Press: New York, N. Y.). A variety of kits are commercially available for extracting nucleic acids (DNA or RNA) from biological samples (e.g., KAPA Express Extract (Roche Sequencing Solutions, Pleasanton, Cal.) and other similar products from BD Biosciences Clontech (Palo Alto, Cal.), Epicentre Technologies (Madison, Wisc.); Gentra Systems, (Minneapolis, Minn.); and Qiagen (Valencia, Cal.), Ambion (Austin, Tex.); BioRad Laboratories (Hercules, Cal.); and more.

The present invention involves detecting an epigenetic modification, specifically, an epigenetic cytosine modification in nucleic acids (including, but not limited to, cytosine methylation). The nucleic acid sequences that are subject to conditional epigenetic modification are the target sequences analyzed by the method disclosed herein. The same nucleic acid sequence may or may not have the epigenetic modification characterized by methylation of cytosines at the 5-position (5mC or 5hmC). In some embodiments, a set or a panel of target nucleic acids are probed for the presence of methylation. For example, as shown in Patai, et al. “Comprehensive DNA Methylation Analysis Reveals a Common Ten-Gene Methylation Signature in Colorectal Adenomas and Carcinomas” PLOS ONE 10(8): e0133836 (2015), and in Onwuka, et al., “A panel of DNA methylation signature from peripheral blood may predict colorectal cancer susceptibility,” BMC Cancer 20, 692 (2020), methylation of biomarkers in a panel of methylation biomarkers is indicative of the presence of colorectal cancer in the patient. Accordingly, testing any known or future panels of methylation biomarkers for prognostic or diagnostic purposes is envisioned with the method disclosed herein. In some embodiments, the entire genome of an organism is probed for the presence of methylation. The method of the instant invention includes detecting methylation in all sites throughout the genome of an organism to diagnose a disease or condition or predisposition to a disease or condition using the sequence analysis and artificial intelligence tools described, e.g., in Shull, et al. “Sequencing the cancer methylome,” Methods Mol Biol. 1238:627-635 (2015).

In some embodiments, the method of detecting epigenetic modifications includes sequencing. The nucleic acid processed as described herein is subjected to sequencing; preferably, massively parallel single molecule sequencing. Analyzing individual molecules by massively parallel sequencing typically requires a separate level of barcoding for sample identification and error correction. The use of molecular barcodes such as described in U.S. Pat. Nos. 7,393,665, 8,168,385, 8,481,292, 8,685,678, and 8,722,368. A unique molecular barcode is added to each molecule to be sequenced to mark molecule and its progeny (e.g., the original molecule and its amplicons generated by PCR). The unique molecular barcode (UID) has multiple uses including counting the number of original target molecules in the sample and error correction (Newman, et al., “An ultrasensitive method for quantitating circulating tumor DNA with broad patient coverage,” Nature Medicine doi: 10.1038/nm.3519 (2014)).

Nanopore sequencing is a unique, scalable technology that enables direct, real-time analysis of long DNA or RNA fragments. It works by monitoring changes to an electrical current as nucleic acids are passed through a protein nanopore. The resulting signal is decoded to provide the specific DNA or RNA sequence. Nanopore-based sequencing technology detects the unique electrical signals of different molecules as they pass through the nanopore with a semiconductor-based electronic detection system. This technology makes for a high throughput, cost effective sequencing solution. At the heart of the technology is the biological nanopore, a protein pore embedded in a membrane, while the brains of the technology lie in the electronics of a semiconductor integrated circuit and proprietary chemistries. The electronic sensor technology embedded in the chip enables automatic membrane assembly and nanopore insertion, while allowing for active control of individual sensors on the circuit. Different sequencing chemistries can be paired with the nanopore and electronic sensor technology to enable high throughput, high accuracy sequencing with faster time-to-data.

In some embodiments, unique molecular barcodes (UIDs) are used for sequencing error correction. The entire progeny of a single target molecule is marked with the same barcode and forms a barcoded family. A variation in the sequence not shared by all members of the barcoded family is discarded as an artefact. Barcodes can also be used for positional deduplication and target quantification, as the entire family represents a single molecule in the original sample (Newman, et al. “Integrated digital error suppression for improved detection of circulating tumor DNA,” Nature Biotechnology 34:547 (2016)).

In some embodiments, the method involves forming a library comprising nucleic acids from a sample. The library consists of a plurality of nucleic acids ready for sequencing or another type of detection method, e.g., PCR. A library can be stored and used multiple times for further processing such as amplification or sequencing of the nucleic acids in the library. In some embodiments, the library is the input nucleic acid in which methylation is detected by the method described herein. In other embodiments, the library is formed from nucleic acids that have undergone the methylation detection reactions described herein.

In some embodiments, the nucleic acids processed for detection of epigenetic modifications according to the method described herein are sequenced. Any of a number of sequencing technologies or sequencing assays can be utilized. The term “Next Generation Sequencing (NGS)” as used herein refers to sequencing methods that allow for massively parallel sequencing of clonally amplified molecules and of single nucleic acid molecules.

Non-limiting examples of sequence assays that are suitable for use with the methods disclosed herein include nanopore sequencing (U.S. Patent Publication Nos. US2013/0244340, US2013/0264207, US2014/0134616, US2015/0119259 and US2015/0337366), Sanger sequencing, capillary array sequencing, thermal cycle sequencing (Sears, et al., Biotechniques, 13:626-633 (1992)), solid-phase sequencing (Zimmerman, et al., Methods Mol. Cell Biol., 3:39-42 (1992)), sequencing with mass spectrometry such as matrix-assisted laser desorption/ionization time-of-flight mass spectrometry (MALDI-TOF/MS; Fu, et al., Nature Biotech., 16:381-384 (1998)), sequencing by hybridization (Drmanac et al., Nature Biotech., 16:54-58 (1998), and NGS methods, including but not limited to sequencing by synthesis (e.g., HiSeq™, MiSeq™, or Genome Analyzer, each available from Illumina), sequencing by ligation (e.g., SOLID™, Life Technologies), ion semiconductor sequencing (e.g., Ion Torrent™, Life Technologies), and SMRT® sequencing (e.g., Pacific Biosciences).

Commercially available sequencing technologies include: sequencing-by-hybridization platforms from Affymetrix Inc. (Sunnyvale, Calif.), sequencing-by-synthesis platforms from Illumina/Solexa (San Diego, Calif.) and Helicos Biosciences (Cambridge, Mass.), sequencing-by-ligation platform from Applied Biosystems (Foster City, Calif.). Other sequencing technologies include, but are not limited to, the Ion Torrent technology (ThermoFisher Scientific), and nanopore sequencing (Genia Technology, Roche Sequencing Solutions, Santa Clara, Cal.), Oxford Nanopore Technologies (Oxford, UK), and sequencing by expansion (Stratos Genomics, Roche Sequncing Solutions).

In some embodiments, the sequencing step involves sequence aligning. In some embodiments, aligning is used to determine a consensus sequence from a plurality of sequences, e.g., a plurality having the same unique molecular ID (UID). The molecular ID is a barcode that can be added to each molecule prior to sequencing or if amplification step is included, prior to the amplification step. In some embodiments, a UID is present in the 5′-portion of the RT primer. Similarly, a UID can be present in the 5′-end of the last barcode subunit to be added to the compound barcode. In other embodiments, a UID is present in an adaptor and is added to one or both ends of the target nucleic acid by ligation.

In some embodiments, a consensus sequence is determined from a plurality of sequences all having an identical UID. The sequences having an identical UID are presumed to derive from the same original molecule through amplification. In other embodiments, UID is used to eliminate artifacts, i.e., variations existing in the progeny of a single molecule (characterized by a particular UID). Such artifacts resulting from PCR errors or sequencing errors can be eliminated using UIDs.

In some embodiments, the number of each sequence in the sample can be quantified by quantifying relative numbers of sequences with each UID among the population having the same multiplex sample ID (MID). Each UID represents a single molecule in the original sample and counting different UIDs associated with each sequence variant can determine the fraction of each sequence variant in the original sample, where all molecules share the same MID. A person skilled in the art will be able to determine the number of sequence reads necessary to determine a consensus sequence. In some embodiments, the relevant number is reads per UID (“sequence depth”) necessary for an accurate quantitative result. In some embodiments, the desired depth is 5-50 reads per UID.

### Modification of 5fC

Modification of 5fC according to the present invention can be achieved if the first reactive group of the reagent according to the present invention is a C-H acidic group, an amine or a phosphorus ylide.

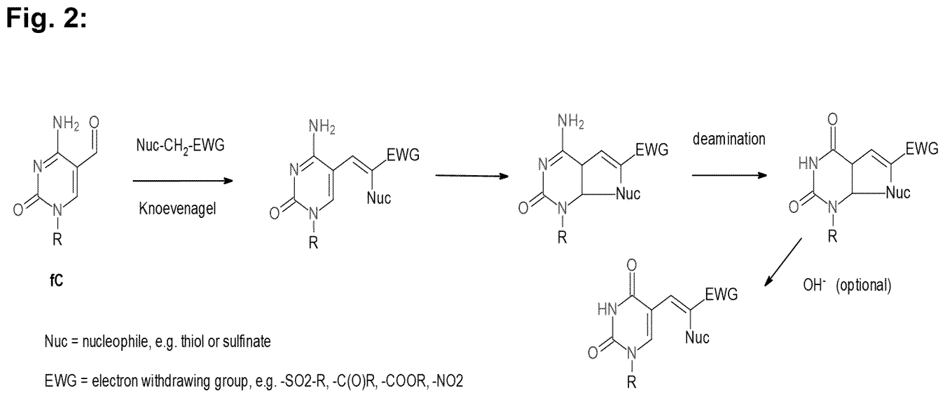

If the first reactive group is a C-H acidic group, the reaction can be executed as a Knoevenagel condensation, which is schematically depicted in FIG. 2. The CH-acidic reagent comprising a nucleophile group is added via Knoevenagel conditions to fC, then subsequent intramolecular addition of the nucleophile to the C5-C6 double bond takes place, thereby enabling the deamination at C4 to obtain a “T-equivalent”. Yields of intramolecular addition of the nucleophile to the C5-C6 double bond can be increased by a photoisomerization of the double bond and/or by applying a radical initiation or a catalyst. If desirable, a reversal of the ring closure can be achieved under alkaline conditions.

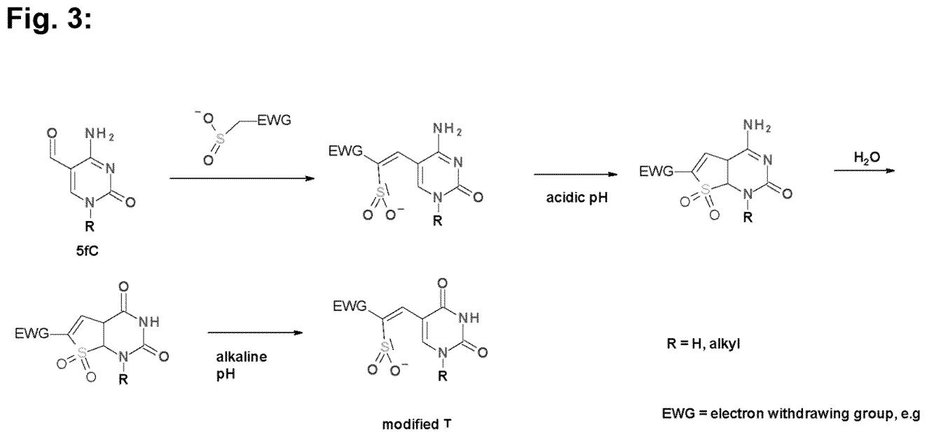

Alternatively, an aldol condensation reaction of 5fC with a CH-acidic reagent comprising a sulfinate moiety (e.g. 1-(methylsulfonyl)methane sulfinate), a subsequent addition of a sulfinate moiety at the C-6 position of the substituted cytosine, followed by deamination and desulfination (5fC to T conversion) can be executed (FIG. 3) A desulfination step under alkaline conditions may even not be required. Instead of a sulfinate moiety, a thiol moiety can also be applied as nucleophile, addition reaction to the C5-C6 double bond can be achieved by means of applying thiol-ene chemistry using a radical initiator or a catalyst. The yields may be increased by photoisomerization of the newly formed double bond.

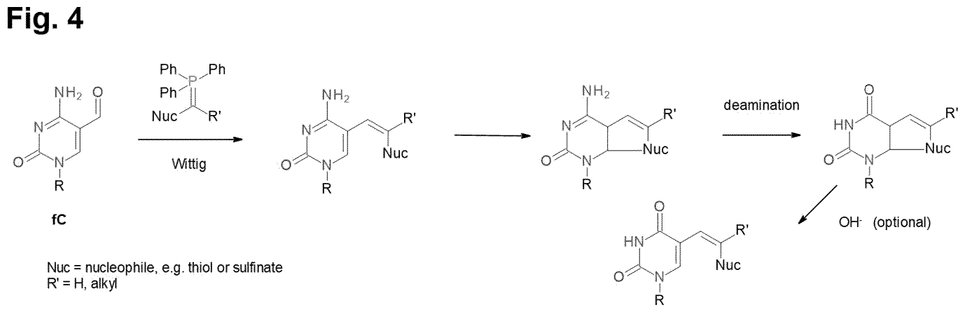

Modification of 5fC according to the present invention can also be done by means of a Wittig reaction (FIG. 4). I this case, the reagent according to the present invention is a phosphorus ylide comprising a nucleophilic group. The reagent is added via Wittig conditions to 5fC, then a subsequent intramolecular addition of the nucleophile to the C5-C6 double bond takes place, thereby enabling a deamination at the C4 position in order to obtain a “T-equivalent”.

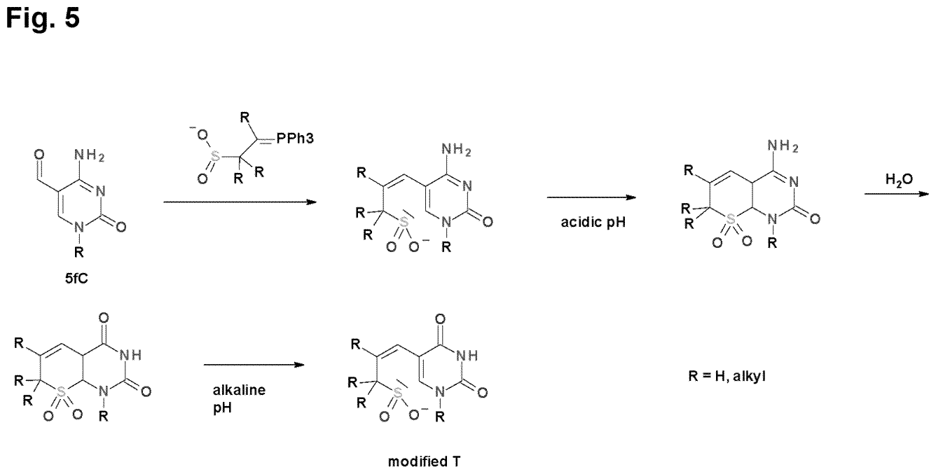

Details of the reaction are shown for a particular example in FIG. 5. In this case, 5fC is reacted with a sufinate modified Wittig reagent. Subsequently there is an addition of the sulfinate moiety at the C-6 position of substituted cytosine, followed by a deamination and desulfination (5fC to T conversion). The desulfination step under alkaline conditions may not be needed necessarily. Alternatively, instead of a sulfinate moiety, a thiol moiety can also be used by means of applying thiol-ene chemistry, using a radical initiator or a catalyst. Yields of intramolecular addition of the nucleophile to the C5-C6 double bond may be increased by photoisomerization of the newly formed double bond, and/or by applying radical initiation or a catalyst. If desirable, a reversal of the ring closure can be achieved under alkaline conditions. Alternatively, the reaction can be performed using Horner-Wadsworth-Emmons reaction conditions, in particular using a sulfinate group as nucleophile.

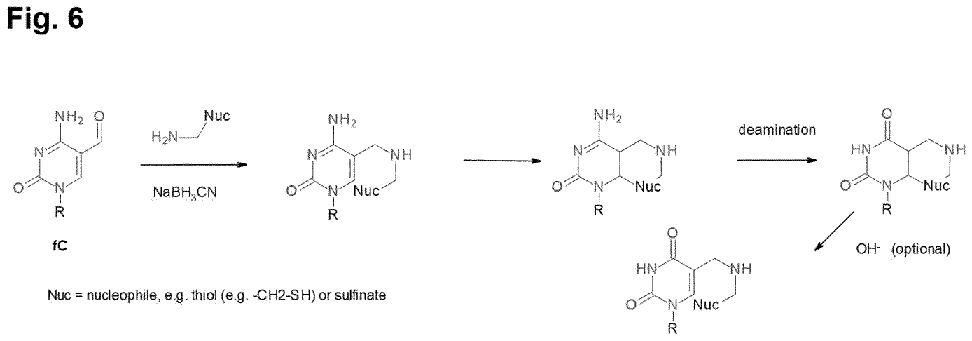

Yet another alternative is the modification of 5fC by means of reductive amination as shown in FIG. 6. In this case, the reagent of the invention comprising an amine and a further nucleophilic group is added via reductive amination to 5fC. Subsequently there is an intramolecular addition of the nucleophile to the C5-C6 double bond, which enables the deamination at the C4 position to obtain a “T-equivalent”. Reversal of ring closure can be achieved under alkaline conditions, if necessary. Yields can be increased by applying radical initiation or a catalyst.

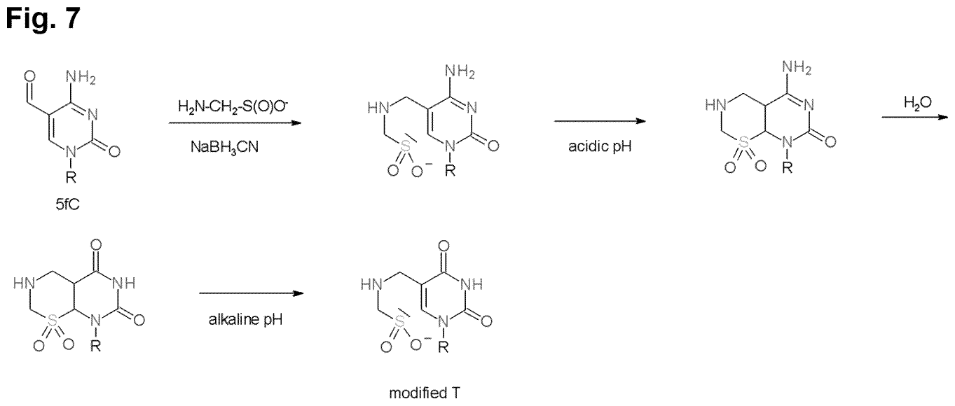

A more detailed example for implementing a reductive amination reaction is illustrated in FIG. 7. 5fC is reacted with 2-aminomethyl sulfinate, and subsequently addition of the sulfinate moiety at C-6 position of substituted cytosine takes place. This is followed by a deamination and desulfination step, which converts 5fC to T. Again, a desulfination step under alkaline conditions may not be needed. Also again, the sulfinate moiety may be replaced by a thiol moiety using thiol-ene chemistry for the addition reaction to double bond C5-C6, which optionally includes the use of a radical initiator or a catalyst.

### Modification of HmC

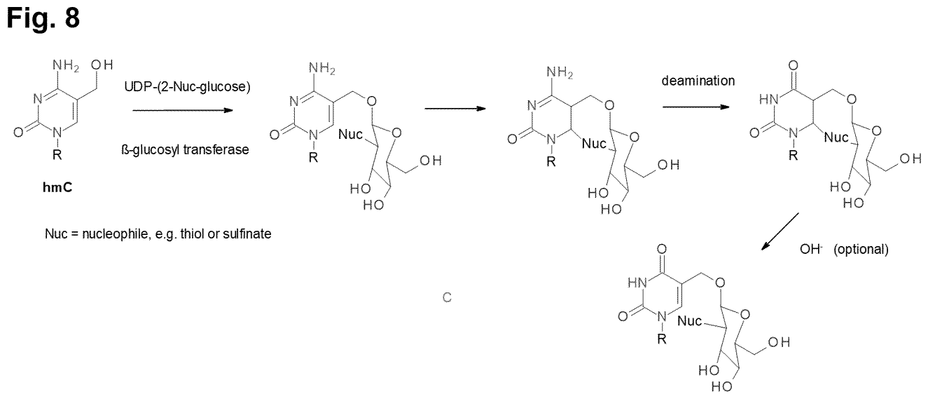

FIG. 8 schematically shows a reaction scheme for the modification of hmC. In this case, the reagent used according to the present invention is a UDP-glucose, which is modified at the 2-position with a nucleophilic group. It is added via a β-glucosyl transferase enzyme to hmC. Then, a subsequent intramolecular addition of the nucleophile to the C5-C6 double bond takes place, thereby enabling the deamination at the C4 position in order to obtain a “T-equivalent”. As discussed above, yields of intramolecular addition of the nucleophile to the C5-C6 double bond can be increased by applying radical initiation or a catalyst and a reversal of the ring closure can be achieved under alkaline conditions, if necessary.

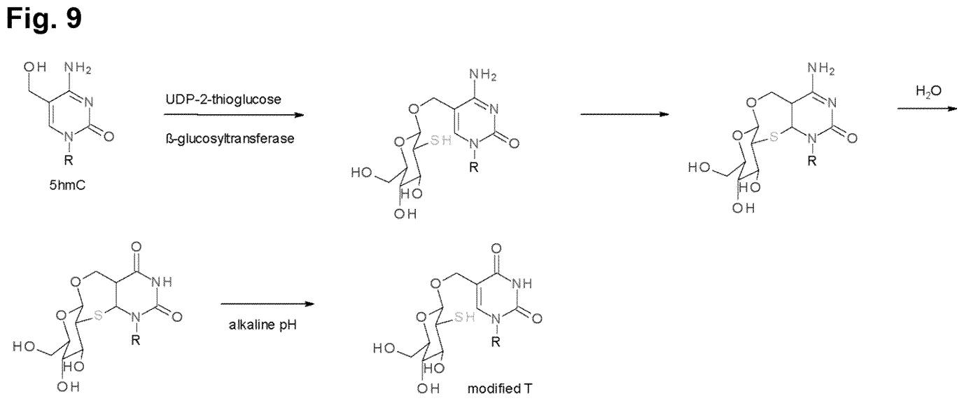

An alternative example is shown in FIG. 9. In this case, 5 hmC is reacted with 2-thio-glucose-UDP and β-glucosyltransferase. Subsequently, an addition of the thiol moiety at the C6 position of substituted cytosine applying thiol-ene chemistry and optionally using a radical initiator or a catalyst is taking place, which is followed by a deamination reaction, resulting in a 5 hmC to T conversion. Alternatively, 2-Sulfinato-glucose-UDP can also be applied as substrate for β-glucosyltransferase.

### Modification of caC

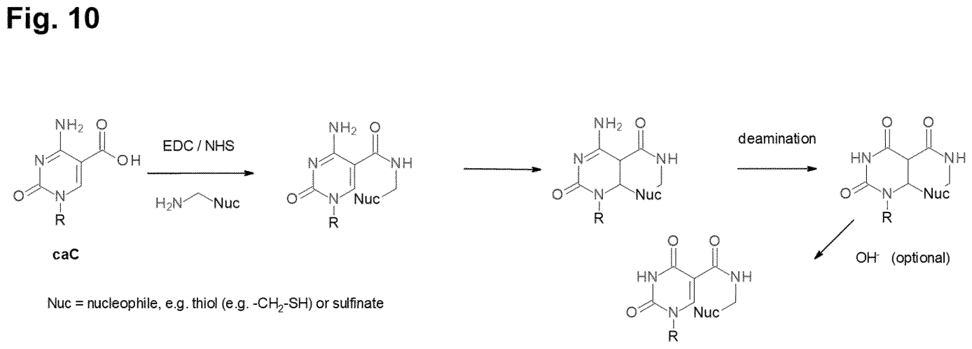

For this embodiment, which is shown in FIG. 10, the method of the present invention uses a reagent comprising an amine and a further nucleophilic group. It is added after an EDC/NHS activation of the carboxyl group of caC. This results in caC forming an amide bond, and a subsequent intramolecular addition of the nucleophile to the C5-C6 double bond, thereby enabling the deamination at the C4 position in order to obtain a “T-equivalent”. Again, the yield of intramolecular addition of the nucleophile to the C5-C6 double bond can be increased by means of applying radical initiation or a catalyst and reversal of the ring closure can be achieved under alkaline conditions.

1. A method, wherein the method comprises the following steps:
(a) providing a nucleic acid comprising 5fC, 5hmC, or 5caC,
(b) providing one reactant, wherein the one reactant comprises two reactive groups, and wherein (i) the first reactive group is capable of reacting with formyl, hydroxymethyl, or carboxyl group, and (ii) the second reactive group is a nucleophilic group,
(c) reacting the first reactive group with the formyl, hydroxymethyl, or carboxyl group thereby resulting in a modified 5fC, 5hmC, or 5caC,
(d) reacting the second reactive group with the C6 position of the modified 5fC, 5hmC, or 5caC, thereby obtaining a bicyclic or tricyclic molecule wherein the bicyclic or tricyclic molecule comproses a 5,6-di-hydro Cystosine entity, and
(e) deaminating the 5,6-di-hydroCytosine entity to a 5,6-di-hydro-Uracil entity.

2. The method of claim 1, further comprising the following step:
(f) reversing the ring formation of step (d), thereby obtaining a 5-substituted Uracil.

3. The method of claim 1, further comprising the step of converting 5mC to 5 fC, 5 hmC or 5caC prior to step (a).

4. The method of claim 1, wherein the nucleic acid comprises 5fC.

5. The method of claim 4, wherein the first reactive group is a C-H acidic group, an amine group, or a phosphorus ylide.

6. The method of claim 1, wherein the nucleic acid comprises 5hmC.

7. The method of claim 6, wherein the first reactive group is a Glycosyl Transferase substrate.

8. The method of claim 1, wherein the nucleic acid comprises 5caC.

9. The method of claim 8, wherein the first reactive group is an amine.

10. The method of claim 1, wherein the nucleophilic group is selected from a group consisting of Thiol or Sulfinate.

11. The method of any one of claims 1-10, further comprising a subsequent step of amplifying the nucleic acid.

12. The method of claim 11, further comprising the subsequent step of sequencing the nucleic acid.

13. A composition comprising a nucleic acid treated according to the method of any one of claims 1-10.

14. A kit for performing the method of any one of claims 1-10.
